(19) United States
(12) Patent Application Publication
(10) Pub. No.: US 2018/0036227 A1
(43) Pub. Date: Feb. 8, 2018

(54) TOPICAL COMPOSITIONS

(71) Applicant: CMPD LICENSING, LLC, Conroe, TX (US)

(72) Inventor: Jay Richard Ray, II, Conroe, TX (US)

(73) Assignee: CMPD LICENSING, LLC, Conroe, TX (US)

(21) Appl. No.: 15/668,184

(22) Filed: Aug. 3, 2017

Related U.S. Application Data
(60) Provisional application No. 62/370,571, filed on Aug. 3, 2016.

(51) Int. Cl. A61K 9/00; A61K 31/17; A61K 8/36; A61K 8/42; A61Q 19/00

(52) U.S. Cl. CPC A61K 9/0014; A61K 8/42; A61Q 19/00; A61K 8/36; A61K 31/17

(57) ABSTRACT

A topical composition that may be a lotion, gel, ointment, foam, cream, or emulsion, and that may include urea and fluocinonide. The topical composition may include between approximately 10% to approximately 25% urea by weight. The topical composition may further include between approximately 0.050% to approximately 0.075% fluocinonide by weight.

### CROSS-REFERENCE TO RELATED APPLICATIONS

The present application claims priority under 35 U.S.C. §119(e) to U.S. Provisional Patent Application No. 62/370,571, filed on Aug. 3, 2016, which is hereby incorporated by reference into this specification.

### TECHNICAL FIELD

The present application is directed to topical compositions. More specifically, the present application is directed to topical compositions containing urea.

### SUMMARY

In one aspect, a topical composition may include urea and fluocinonide. The topical composition may be a lotion, gel, ointment, foam, cream, or emulsion. The topical composition may include between approximately 10% to approximately 25% urea by weight. The topical composition may further include between approximately 0.05% to approximately 0.075% fluocinonide by weight.

### DESCRIPTION

The present disclosure describes topical compositions. The topical compositions may include urea and fluocinonide. In various embodiments, the topical compositions may include a compounded cream that includes urea and fluocinonide. In various embodiments, the topical compositions may include a combination of topical creams, such as a combination of a urea topical cream and a fluocinonide topical cream.

The topical composition may be any type of composition. For example, the topical composition may be or include a lotion, gel, ointment, foam, cream, emulsion, or any other composition intended for application to the skin and/or nails of a patient. Unless otherwise apparent, the term “cream” as used herein includes lotions, gels, ointments, foams, creams, or emulsions. The topical composition may include urea. Urea is an organic compound having the chemical formula CO(NH₂)₂. Urea has the following chemical structure:

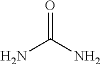

Urea has physiologic importance as a nitrogen carrier and osmolyte. Urea is produced biologically within the urea cycle as nitrogenous compounds or amino acids are metabolized. Industrial production of urea involves conversion of ammonia and carbon dioxide to urea.

Cosmetic and therapeutic products intended for application to skin and nails may incorporate urea. Urea creams for dermatological application have been used to treat dry or scaly skin by promoting skin hydration. The hydration or rehydration related properties of urea are also believed to aid in skin penetration. Remeven™ marketed by Stratus Pharmaceuticals Inc., FL, is an example of a urea cream including 50% urea. Rea Lo 30® or Rea Lo 40® marketed by Crown Laboratories, TN, are additional examples of urea creams that are keratolytic emollients used as skin softeners. Indicated uses for topical urea creams include eczema, ichthyosis, and dermatitis conditions. Topical urea has also been used as a penetrant agent in conjunction with other medications. U-Cort®, marketed by Taro Pharmaceuticals Inc., NY, is a 1% hydrocortisone acetate cream containing urea indicated for relief of inflammatory and pruritic manifestations of corticosteroid-responsive dermatoses.

The topical composition may include any amount of urea by weight in the topical composition. In various embodiments, the amount of urea by weight in the topical composition may be between approximately 10% and approximately 25%, between approximately 15% and approximately 25%, between approximately 20% and approximately 25%, between approximately 10% and approximately 20%, between approximately 10% and approximately 15%, or between approximately 5% and approximately 30%. In these or other embodiments, the amount of urea by weight in the topical composition may be approximately 10%, approximately 11%, approximately 12%, approximately 14%, approximately 15%, approximately 16%, approximately 18%, approximately 20%, approximately 22%, approximately 24%, approximately 25%, or any other percentage (including any percentage between approximately 5% and approximately 30%).

In various embodiments, the urea may be included in the topical composition in the form of a commercially available urea cream. For example, a component of the topical composition may be a commercial urea cream providing all or a portion of the urea in the topical composition. The urea cream may comprise various percentages of urea by weight (prior to compounding or prior to combination with another cream), such as 10%, 15%, 20%, 25%, 30%, 35%, 40%, or any other commercially available percentage by weight.

In various embodiments, the urea cream may be Urix 40 Urea Cream marketed by Topix Pharmaceuticals, Inc. Urix 40 Urea Cream includes 40% urea or 400 mg urea per gram and further includes Carbomer, Cyclomethicone, Dimethicone Silyate, Dimethiconol, Glycerin, Hydrogenated Lecithin, Imidazolidinyl Urea, Petrolatum, Phenyl Trimethicone, Polyphosphorylcholine Glycol Acrilate, Triethanolamine, Water, and Xanthan Gum. In additional embodiments, the urea cream may be Rea Lo 40 topical or Rea Lo 30 topical marketed by Crown Laboratories. Rea Lo 40 topical comprises 400 mg urea per gram and Rea Lo 30 topical comprises 300 mg urea per gram. Rea Lo 40 topical and Rea Lo 30 topical further include purified water, emulsifying wax, glycerin, isopropyl myristate, sorbitol, neopentyl, glycol dicaprylate/dicaprate, tridecyl stearate, tridecyl trimellitate and dimethyl isosorbide. In additional embodiments, the urea cream may be Urea 10% Cream by Stratus Pharmaceuticals, Inc. Urea 10% Cream includes 10% urea or 100 mg urea per gram, and further includes Carbomer, Fragrance, Isopropyl Myristate, Isopropyl Palmitate, Propylene Glycol, Purified Water, Sodium Laureth Sulfate, Stearic Acid, Trolamine and Xanthan Gum.

It is to be understood that the above urea creams (or any other urea cream) may be diluted or cut prior to or, in some embodiments, after compounding or otherwise combining the urea cream with additional creams and/or actives. Thus, the topical composition may comprise less urea by weight than was present in the urea cream prior to compounding or combination with another cream and/or active.

In various embodiments, the urea may be included in the topical composition in the form of a compounded urea cream. For example, urea may be added to a base (such as a commercially available base) in order to form a compounded urea cream. The base may be a foam, cream, gel, lotion, ointment, or emulsion (oil-in-water or water-in-oil), for example, suitable for topical application, e.g., to skin or nails. For brevity, such bases are referred to herein as cream bases or base creams. Unless otherwise specified, a cream base or a base cream as used herein may include bases that are foams, gels, lotions, ointments, creams, or emulsions (oil-in-water or water-in-oil).

In some embodiments, the cream base includes polyethylene glycol (PEG). In other embodiments, the cream base is PEG-free. In these or other embodiments, the cream base may include a silicon or silicon variant. In some embodiments, the cream base is silicon-free. An example cream base comprising a foam may include a propellant such as butane. Cream bases comprising a foam may also comprise additional characteristics such as that of an emulsion, such as an oil-in-water emulsion. In one example, the cream base comprises an ointment, e.g., water soluble/miscible, absorption, water-in-oil emulsion, or oil-in-water emulsion. Example cream bases comprising ointments may include hydrophilic petrolatum, white tetrolatum, hydrophilic ointment, white ointment, anhydrous lanolin, hydrous lanolin, PEG ointment, or combinations thereof.

The cream base may comprise various emollients. For example, in one embodiment the cream base comprises a keratolytic emollient. In one embodiment, the cream base may comprise one or more of acrylates copolymer, alcohol, camphor, carbomer, dimethyl isosorbide, disodium EDTA, dl-alphatocopheryl acetate, edetate disodium, emulsifying wax, eucalyptus oil, flavonoids, glycerin, glycol dicaprylate/dicaprate, hydroxyethyl cellulose, isopropyl myristate, lactic acid, meadowsweet extract, menthol, mineral oil, neopentyl, phenolic glycosides, polyethylene glycol (PEG), polysorbate 85, purified water, titanium dioxide, tridecyl stearate, tridecyl trimellitate, sodium hydroxide, sodium hydroxide, sorbitol, stearic acid, or zinc pyrithione. In various embodiments, the cream base may be a commercially available topical base. For example, in some embodiments, the cream base may be the topical base Spira-Wash™ Gel or Lipoderm® both marketed by Professional Compounding Centers of America (PCCA), Houston, Tex.

The urea may be added to the base in any manner to form the compounded urea cream. As an example, the urea may be mixed into the base. Also, any amount of the urea may be added to the base to form the compounded urea cream. For example, an amount of urea may be added to the base so as to cause the compounded urea cream to include various percentages of urea by weight (prior to combination with another cream) such as 10%, 15%, 20%, 25%, 30%, 35%, 40%, or any other percentage.

In addition to urea, the topical composition may further include fluocinonide. Fluocinonide is a potent glucocorticoid having the chemical formula C₂₆H₃₂F₂O₇, and the following chemical structure:

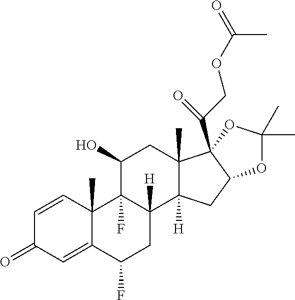

The topical composition may include any amount of fluocinonide by weight in the topical composition. In various embodiments, the amount of fluocinonide by weight in the topical composition may be between approximately 0.050% and approximately 0.075%, approximately 0.055% and approximately 0.075%, approximately 0.060% and approximately 0.075%, approximately 0.065% and approximately 0.075%, approximately 0.070% and approximately 0.075%, approximately 0.050% and approximately 0.070%, approximately 0.050% and approximately 0.065%, approximately 0.050% and approximately 0.060%, approximately 0.050% and approximately 0.055%, or approximately 0.020% and approximately 0.095%. In these or other embodiments, the amount of fluocinonide by weight in the topical composition may comprises approximately 0.050%, approximately 0.055%, approximately 0.060%, approximately 0.065%, approximately 0.070%, approximately 0.075%, or any other percentage (including any percentage between approximately 0.020% and approximately 0.095%).

In various embodiments, the fluocinonide may be included in the topical composition in the form of a commercially available fluocinonide cream. For example, a component of the topical composition may be a commercial fluocinonide cream providing all or a portion of the fluocinonide in the topical composition. The fluocinonide creams may include various percentages of fluocinonide by weight prior to compounding such as 0.025%, 0.050%, 0.075%, 0.1%, 0.2%, or any other percentage.

In various embodiments, the fluocinonide cream may be Fluocinonide Cream USP, 0.1% manufactured by E. Fougera & Co. Each gram of fluocinonide cream USP, 0.1% contains 1 mg fluocinonide in a cream base of propylene glycol USP, diethylene glycol monoethyl ether NF, glyceryl stearate (and) PEG-100 stearate, purified water USP, glyceryl monostearate NF, white petrolatum USP, carbomer 980 NF, diisopropanolamine, and citric acid USP. In additional embodiments, the fluocinonide cream may be Vanos® (fluocinonide) Cream, 0.1% marketed by Medicis Pharmaceutical Corp. Each gram of Vanos Cream contains 1 mg micronized fluocinonide in a cream base of propylene glycol USP, dimethyl isosorbide, glyceryl stearate (and) PEG-100 stearate, glyceryl monostearate NF, purified water USP, carbopol 980 NF, diisopropanolamine, and anhydrous citric acid USP. In additional embodiments, the fluocinonide cream may be Lidex (fluocinonide) Cream 0.05% marketed by Medicis, The Dermatology Company®. Lidex Cream contains fluocinonide 0.5 mg/g in FAPG® cream, and a specially formulated cream base consisting of citric acid, 1,2,6-hexanetriol, polyethylene glycol 8000, propylene glycol and stearyl alcohol.

It is to be understood that the above fluocinonide creams (or any other fluocinonide cream) may be diluted or cut prior to or, in some embodiments, after compounding or otherwise combining the fluocinonide cream with additional creams and/or actives. Thus, the topical composition may comprise less fluocinonide by weight than was present in the fluocinonide cream prior to compounding or combination with another cream and/or active.

In various embodiments, the fluocinonide may be included in the topical composition in the form of a compounded fluocinonide cream. For example, fluocinonide may be added to a base (such as a commercially available base) in order to form a compounded fluocinonide cream. The base may be any of the bases discussed above with regard to the compounded urea cream.

The fluocinonide may be added to the base in any manner to form the compounded fluocinonide cream. As an example, the fluocinonide may be mixed into the base. Also, any amount of the fluocinonide may be added to the base to form the compounded fluocinonide cream. For example, an amount of fluocinonide may be added to the base so as to cause the compounded fluocinonide cream to include various percentages of fluocinonide by weight (prior to combination with another cream) such as 0.025%, 0.050%, 0.075%, 0.1%, 0.2%, or any other percentage (including any percentage between 0.025% and 0.2%).

As is discussed above, the topical composition may include urea and fluocinonide. In various embodiments, the topical composition may include an amount of urea by weight of approximately 12% and an amount of fluocinonide by weight of approximately 0.075%. In various embodiments, the topical composition may include an amount of urea by weight of approximately 13.3% and an amount of fluocinonide by weight of approximately 0.067%. In various embodiments, the topical composition may include an amount of urea by weight of approximately 20% and an amount of fluocinonide by weight of approximately 0.05%.

The urea and the fluocinonide may be included in the topical composition in any form. In various embodiments, the urea may be included in the topical composition in the form of a commercially available urea cream, and the fluocinonide may be included in the topical composition in the form of a commercially available fluocinonide cream. In various embodiments, the urea may be included in the topical composition in the form of a compounded urea cream, and the fluocinonide may be included in the topical composition in the form of a compounded fluocinonide cream. In various embodiments, the urea may be included in the topical composition in the form of a commercially available urea cream, and the fluocinonide may be included in the topical composition in the form of a compounded fluocinonide cream. In various embodiments, the urea may be included in the topical composition in the form of a compounded urea cream, and the fluocinonide may be included in the topical composition in the form of a commercially available fluocinonide cream.

### Methods

The topical composition may comprises a combination of topical creams for sequential application to affected skin or a compounded topical cream formulated for topical administration to affected skin, such a hands, feet, elbows, knees, limbs, scalp, torso, etc. Example dosage forms may include creams, lotions, gels, oils, or ointments, or any other topical forms known to those skilled in the art. Methods of using the topical composition may include treating an individual in need by topically applying the cream to affected skin. Conditions treated may include conditions such as those marked by skin that is one or more of dry, scaly, thick, calloused, hardened, or rough. Example conditions may include allergies, dermatitis, psoriasis, rashes, etc. The topical composition described herein may thus be applied to such affected areas of the skin to treat the affected area. The cream may alleviate symptoms such as redness, swelling, or itching. The cream may accelerate the healing process with respect to the affected skin. In various embodiments, the topical composition may be administered to treat hyperkeratotic conditions.

A method of making the topical composition comprising a compounded topical cream including urea and fluocinonide, as described herein, may include compounding a first topical cream and a second topical cream. The first topical cream may be a urea cream and the second topical cream may be a fluocinonide cream. For example, the method of making the compounded topical cream may comprise combining a urea cream, such as a commercially available urea 10% cream, urea 20% cream, urea 30% cream, urea 40% cream, or other suitable urea cream, with a fluocinonide cream, such as a commercially available fluocinonide 0.1% cream or other suitable fluocinonide cream. In one embodiment, the method may include compounding the creams, such as by cold compounding. The method may further include combining one or more additional active or inactive agents.

The method of making the topical composition may comprise selecting a urea cream. The urea cream may be a urea cream containing various percentages by weight of urea prior to combination administration or compounding such as 5%, 10%, 15%, 20%, 25%, 30%, 35%, 40%, 45%, 50%, 55%, or greater. In one embodiment, the urea cream is a commercially available urea cream. It is to be understood that urea creams may be further supplemented with urea, diluted, or cut prior to or, in some embodiments, after compounding the urea cream with the fluocinonide cream or one or more additional active ingredients or inactive ingredients. Thus the topical composition may comprise more or less urea by weight than was present in the urea cream prior to combination, administration, or compounding. Urea cream may be combined with additional components of the topical composition in an amount such that the resultant cream comprises between approximately 10% and approximately 25%, between approximately 15% and approximately 25%, between approximately 20% and approximately 25%, between approximately 15% and approximately 20%, between approximately 10% and approximately 20%, or between approximately 10% and approximately 15% urea by weight.

The method of making the topical composition may further comprise selecting a fluocinonide cream. The fluocinonide cream may be a fluocinonide cream containing various percentages by weight of fluocinonide prior to combination, administration, or compounding, such as 0.1% or other suitable amount. In one embodiment, the fluocinonide cream is a commercially available fluocinonide cream. It is to be understood that fluocinonide creams may be further supplemented with fluocinonide, diluted, or cut prior to or, in some embodiments, after compounding the fluocinonide cream with the urea cream or one or more additional active ingredients or inactive ingredients. Thus, the topical composition may comprise more or less fluocinonide by weight than was present in the fluocinonide cream prior to combination, administration, or compounding. Fluocinonide cream may be combined with components of the topical composition in an amount such that the combination topical creams or compounded topical cream comprises between approximately 0.05% and approximately 0.075%, between approximately 0.055% and approximately 0.075%, between approximately 0.06% and approximately 0.075%, between approximately 0.065% and approximately 0.075%, between approximately 0.07% and approximately 0.075%, between approximately 0.05% and approximately 0.06%, or between approximately 0.06% and approximately 0.07%% fluocinonide by weight.

Accordingly, the method may comprise combining or compounding urea cream and fluocinonide cream, both of which may be commercially available topical creams, to formulate a combination of topical creams or a compounded topical cream comprising between approximately 10% and approximately 25% urea by weight and between approximately 0.05% and approximately 0.075% fluocinonide by weight. In one embodiment, at least a portion of the urea and at least a portion of the fluocinonide are provided by the combination or addition of the respective commercial topical creams to the compounded topical cream. For example, a method of making a compounded topical cream comprising approximately 13% urea and approximately 0.067% fluocinonide by weight includes combining 1 part urea 40% cream (e.g., 2 g urea 40% cream) and 2 parts fluocinonide 0.1% cream (e.g., 4 g fluocinonide 0.1% cream). As another example, a method of making 6 g of a compounded topical cream comprising approximately 12% urea and approximately 0.07% fluocinonide by weight includes combining 1.8 g urea 40% cream and 4.2 g fluocinonide 0.1% cream. In another embodiment, the urea component of the topical composition may be provided by a bulk source urea or other that commercial urea cream source.

As noted above, the topical composition may also contain urea from sources other than or instead of urea cream, such as from bulk powders or solutions. Thus, the amount of urea cream or other source urea may be adjusted to obtain the final weight percentages of urea in topical composition. Similarly, the compounded cream may also contain fluocinonide from sources other than fluocinonide cream, such as from bulk powders or solutions. Thus, the amount of fluocinonide cream or other source fluocinonide may be adjusted to obtain the final weight percentages of fluocinonide in topical composition.

The method of making the compounded topical cream may further comprise an addition of one or more additional inactive agents, e.g., emollients, or active agents. In combination of topical creams embodiments, additional active agents may be combined with urea cream, fluocinonide cream, a separate cream, or combinations thereof. In various embodiments, additional active agents may together comprise between approximately 1% to approximately 15% by weight of the topical composition. Additional active agents may be selected from an antibacterial agent, an antiviral agent, an antifungal agent, an antibiotic agent, an anti-inflammatory agent, an non-steroidal anti-inflammatory (NSAID) agent, an anti-allergic agent, an anti-infective agent, an anti-depressant agent, a steroid agent, a stimulant agent, a disinfectant agent, an anticonvulsant agent, a local anesthetic agent, or combinations thereof.

In one embodiment wherein the topical composition includes an additional active agent, the composition comprises between approximately 0.5% and 2.5% by weight of a steroid agent selected from fluticasone, triamcinolone, betamethasone, dexamethasone, flunisolide, prednisone, prednisolone, methylprednisolone, fluocinolone, diflorasone, halcinonide, desoximetasone, diflucortolone, flucloronide, fluocortolone, fluprednidene, flurandrenolide (flurandrenolone), clobetasol, clobetasone, alclometasone, flumethasone, fluocortolone, amcinonide, beclometasone, difluprednate, prednicarbate, flurandrenolide, mometasone, desonide, or combinations thereof. In one embodiment, the steroid agent is or comprises fluticasone wherein the cream comprises approximately 0.5% to approximately 1.5% by weight fluticasone.

In a further embodiment or another embodiment wherein the topical composition includes an additional active agent, the composition comprises between approximately 1.5% to approximately 6.0% by weight of either or both of (a) an antifungal agent comprising an azole selected from itraconazole, clotrimazole, econazole, oxiconazole, ketoconazole, miconazole, voriconazole, sulconazole, or fluconazole, ciclopirox, amphotericin B, Nystatin, terbinafine, amorolfine, flucytosine, or combinations thereof, and (b) an antibiotic agent selected from amikacin, amoxicillin, gentamicin, kanamycin, neomycin, netilmicin, paromomycin, tobramycin, geldanamycin, herbimycin, carbacephem (loracarbef), ertapenem, doripenem, imipenem, cefadroxil, cefazolin, cefalotin, cephalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, clarithromycin, clavulanic acid, clindamycin, colistimethate teicoplanin, azithromycin, dirithromycin, erythromycin, troleandomycin, telithromycin, aztreonam, ampicillin, azlocillin, bacampicillin, carbenicillin, cloxacillin, dicloxacillin, flucloxacillin, mezlocillin, meticillin, nafcillin, norfloxacin, oxacillin, penicillin G, penicillin V, piperacillin, pvampicillin, pivmecillinam, ticarcillin, bacitracin, colistin, colimycin, polymyxin B, ciprofloxacin, enoxacin, gatifloxacin, levofloxacin, lomefloxacin, moxifloxacin, ofloxacin, trovafloxacin, grepafloxacin, sparfloxacin, afenide, prontosil, sulfacetamide, metronidazole, sulfamethizole, sulfanilimide, sulfamethoxazole, sulfisoxazole, trimethoprim, trimethoprim-sulfamethoxazole, demeclocycline, doxycycline, oxytetracycline, tetracycline, arsphenamine, chloramphenicol, chlorhexidine, lincomycin, ethambutol, fosfomycin, furazolidone, isoniazid, linezolid, mupirocin, nitrofurantoin, platensimycin, pyrazinamide, quinupristin/dalfopristin, rifampin, thiamphenicol, rifampicin, minocycline, sultamicillin, sulbactam, sulphonamides, mitomycin, spectinomycin, spiramycin, roxithromycin, meropenem, or combination thereof. In one embodiment, the topical composition comprises between approximately 2.0% to approximately 5.0% by weight of an antifungal agent, wherein the antifungal agent is or comprises the antifungal active itraconazole. In this or another formulation, the antibiotic agent is or comprises the antibiotic active mupirocin wherein the topical composition comprises between approximately 2.0% to approximately 5.0% by weight antibiotic agent.

In a further embodiment or another embodiment wherein the compounded composition includes an additional active agent, the composition comprises approximately 0.5% to approximately 5.0% by weight of either or both of (a) a local anesthetic agent selected from lidocaine, amethocaine, or combinations thereof, and (b) a non-steroidal anti-inflammatory (NSAID) agent selected from indomethacin, ibuprofen, dexibuprophen, diclofenac, ketoprofen, flurbiprofen, piroxicam, pranoprofen lidocaine, amethocaine, benzocaine, prilocaine, or combinations thereof.

In embodiments comprising additional active or inactive agents, e.g., in addition to the urea cream and fluocinonide cream, the amounts of urea cream and fluocinonide cream combined may be adjusted to achieve the desired percent by weight of urea and fluocinonide in the combined or compounded topical cream.

The topical composition may be packaged in suitable containers, such as tubes, for dispensing. In one example, the compounded topical cream may be packaged in 120 gram tubes. The compounded topical cream may be administered as needed, once a day, twice a day, or more. In one embodiment, a 6 g dose of a compounded topical cream comprising 1 part urea 40% cream and 2 parts fluocinonide cream may be applied twice daily.

In one embodiment, a combination of topical creams may be dispensed in a kit for sequential application or mixing prior to application. The kit may include urea cream and fluocinonide cream. The kit may include, for example, one or more tubes of urea cream and one or more tubes of fluocinonide cream, one or both of which may be a commercially available cream. Additional actives or inactives may also be included in the kit.

The user may be directed to apply the topical creams to an affected area sequentially, e.g., one after the other, or together in a compounded topical cream, following mixing. In one embodiment, sequentially includes immediately applying one of the topical creams after the other, such as within approximately 5 minutes. The urea cream may be applied first. For example, a user may be instructed to apply the urea cream to the affected area and then apply the fluocinonide cream to the affected area over the urea cream. In some embodiments, fluocinonide cream may be applied first. A dressing may be applied over the application area. A dressing may be provided in the kit.

In embodiments wherein the user is directed to combine the urea cream and the fluocinonide cream prior to use, the kit may include a container for mixing the topical creams. A stirrer may also be included. For example, the kit may include an ointment jar and a sterile swab for mixing the topical creams prior to use.

A method of dispensing the combination of topical creams may include dispensing the urea cream and fluocinonide cream to a user, which may be included in a kit, as described above. The urea cream and fluocinonide cream may be individual packages for convenient mixing. For example, the urea cream and fluocinonide cream may be provided in single dose tubes. The tubes may contain premeasured amounts of the topical creams thereby allowing the user to combine the proper weight percent of each of urea cream and fluocinonide cream when mixing a dose or sequentially applying the creams to the affected skin. In some embodiments, the tubes may contain multiple doses. In a further embodiment, each of the topical creams is provided in a tube that includes a plunger, e.g., configured similar to a syringe, wherein a user may actuate the plunger to a marked position on the tube to eject an amount of the contained topical cream from the tube for formulating the dose.

In various embodiments, the topical composition comprises a commercially available fluocinonide 0.1% cream and a commercially available urea cream, including a lotion, of any strength dispensed together as non-compounded stand-alone medication to address hyperkeratotic conditions. In one embodiment, the topical composition comprises a commercially available fluocinonide 0.1% cream compounded (including blended) with urea of any strength or form (bulk urea or commercially available urea utilized) and dispensed in a compounded formation to address hyperkeratotic conditions.

This specification has been written with reference to various non-limiting and non-exhaustive embodiments. However, it will be recognized by persons having ordinary skill in the art that various substitutions, modifications, or combinations of any of the disclosed embodiments (or portions thereof) may be made within the scope of this specification. Thus, it is contemplated and understood that this specification supports additional embodiments not expressly set forth in this specification. Such embodiments may be obtained, for example, by combining, modifying, or reorganizing any of the disclosed steps, components, elements, features, aspects, characteristics, limitations, and the like, of the various non-limiting and non-exhaustive embodiments described in this specification. In this manner, Applicant reserves the right to amend the claims during prosecution to add features as variously described in this specification, and such amendments comply with the requirements of 35 U.S.C. §§112(a) and 132(a).

The grammatical articles “one”, “a”, “an”, and “the”, as used in this specification, are intended to include “at least one” or “one or more”, unless otherwise indicated. Thus, the articles are used in this specification to refer to one or more than one (i.e., to “at least one”) of the grammatical objects of the article. By way of example, “a component” means one or more components, and thus, possibly, more than one component is contemplated and may be employed or used in an application of the described embodiments. Further, the use of a singular noun includes the plural, and the use of a plural noun includes the singular, unless the context of the usage requires otherwise. Additionally, the grammatical conjunctions “and” and “or” are used herein according to accepted usage. By way of example, “x and y” refers to “x” and “y”. On the other hand, “x or y” refers to “x”, “y”, or both “x” and “y”, whereas “either x or y” refers to exclusivity.

The present disclosure may be embodied in other forms without departing from the spirit or essential attributes thereof and, accordingly, reference should be had to the following claims rather than the foregoing specification as indicating the scope of the invention. Further, the illustrations of arrangements described herein are intended to provide a general understanding of the various embodiments, and they are not intended to serve as a complete description. Many other arrangements will be apparent to those of skill in the art upon reviewing the above description. Other arrangements may be utilized and derived therefrom, such that logical substitutions and changes may be made without departing from the scope of this disclosure.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims.

What is claimed is:

1. A topical composition comprising a lotion, gel, ointment, foam, cream, or emulsion, the topical composition comprising:
between approximately 10% to approximately 25% urea by weight; and
between approximately 0.05% to approximately 0.075% fluocinonide by weight.

2. The topical composition of claim 1, wherein the urea is in the form of a commercially available urea cream.

3. The topical composition of claim 2, wherein the commercially available urea cream includes a urea 10% cream, urea 20% cream, urea 30% cream, or urea 40% cream.

4. The topical composition of claim 1, wherein the urea is in the form of a compounded urea cream.

5. The topical composition of claim 4, wherein the compounded urea cream includes between approximately 10% and 40% urea.

6. The topical composition of claim 1, wherein the fluocinonide is in the form of a commercially available fluocinonide cream.

7. The topical composition of claim 6, wherein the commercially available fluocinonide cream comprises a fluocinonide 0.025% cream, fluocinonide 0.050% cream, fluocinonide 0.075% cream, fluocinonide 0.1% cream, or fluocinonide 0.2% cream.

8. The topical composition of claim 1, wherein the fluocinonide is in the form of a compounded fluocinonide cream.

9. The topical composition of claim 8, wherein the compounded fluocinonide cream includes between approximately 0.025% and approximately 0.2% fluocinonide.
